(19) United States
(12) Patent Application Publication
(10) Pub. No.: US 2023/0053138 A1
(43) Pub. Date: Feb. 16, 2023

(54) FEED ADDITIVE FOR IMPROVING PERFORMANCE OF DOMESTICATED ANIMALS

(71) Applicant: PMI Nutrition, LLC, Arden Hills, MN (US)

(72) Inventors: David M. Bravo, Yverdon-les-bains (CH); Karin Berger Büter, Uttwil (CH); Bernd Büter, Uttwil (CH)

(21) Appl. No.: 17/874,928

(22) Filed: Jul. 27, 2022

Related U.S. Application Data
(60) Provisional application No. 63/226,668, filed on Jul. 28, 2021.

(51) Int. Cl. A61K 31/341; A61K 9/00; A61K 36/185; A61P 3/02

(52) U.S. Cl. CPC A61K 31/341; A61K 9/0056; A61K 36/185; A61P 3/02; A61K 2236/37

(57) ABSTRACT

The present disclosure provides a feed additive for domesticated animals that is capable of increasing feed performance, as well as associated methods for promoting growth and zootechnical performance in domesticated animals. The animal feed additive provided contains Total Lignans standardized in the contents of Furoguaiacin-like compounds and Guaiacin-like compounds. The present disclosure also describes methods for improving growth via the dietary inclusion of Total Lignans standardized in Furoguaiacin-like compounds and Guaiacin-like compounds.

### REFERENCE TO RELATED APPLICATION

This application claims the benefit of U.S. Provisional Application No. 63/226,668, filed Jul. 28, 2021, which is herein incorporated by reference in its entirety.

### FIELD OF THE INVENTION

The present disclosure relates to the field of animal feed additives, and more specifically to compositions and methods for improving performance of domesticated animals.

### BACKGROUND OF THE INVENTION

Responding to consumers escalating expectations as well as public health concerns, antibiotics, previously massively used in animal feed, are now banned in many countries. Some countries also enacted other limitations on use of therapeutic drugs at the farm level. Such limitations have arisen in view of numerous problems associated with the use of antibiotics or other therapeutics in a farm setting, including antibiotic resistance and the potential for such ingredients to find their way into the food chain. While efforts have been made to explore use of non-traditional feedstuffs in the farm animal diet, these changes have resulted in a diet that is much more problematic than in the past. There is therefore a great need in the art for development of feed additives that could supplant the need for antibiotics and other ingredients while improving performance of farm animals in a drug free context.

### SUMMARY

The present disclosure provides, in one embodiment, an animal feed ingredient composition comprising an effective amount of furoguaiacin or furoguaiacin-like compounds or guaiacin or guaiacin-like compounds, or biologically acceptable salts thereof, wherein the effective amount improves the performance of an animal feed to which the ingredient is added. As used herein, “animal” refers to all animals except humans. Examples of animals are non-ruminants, and ruminants. Ruminant animals include, for example, animals such as sheep, goats, cattle, e.g. beef cattle, dairy cows, cows, and young calves, deer, camel, llama and kangaroo. Non-ruminant animals include mono-gastric animals, e.g. pigs or swine (including, but not limited to, piglets, growing pigs, and sows); poultry such as turkeys, ducks and chickens (including but not limited to broiler chicks, layers); horses (including, but not limited to, hot bloods, cold bloods and warm bloods), fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). Included among such animals that may find use in accordance with the invention are pets, including, but not limited to, dogs and cats.

Biologically acceptable salts that may find use in the present disclosure include, but are not limited to, acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate and p-toluenesulphonic acid, alkali metal salts, with bases, examples of which include the sodium and potassium salts.

In certain embodiments, an animal feed provided herein comprises at least one animal feed component selected from the group consisting of a vitamin, a mineral, a probiotic, an enzyme, a flavoring, an amino acid, and a preservative. Non-limiting examples of a vitamin include, for example, fat-soluble vitamins including vitamin A, vitamin D3, vitamin E, and vitamin K, e.g., vitamin K3; and water-soluble vitamins including vitamin B12, biotin and choline, vitamin B1, vitamin B2, vitamin B6, niacin, folic acid and panthothenate, e.g., Ca-D-panthothenate, and combinations thereof. Non-limiting examples of a mineral include, for example, calcium, magnesium, potassium and sodium, and trace minerals include boron, cobalt, chloride, chromium, copper, fluoride, iodine, iron, manganese, molybdenum, selenium and zinc. Non-limiting examples of a probiotic include, for example, Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof. Non-limiting examples of an enzyme include, for example, acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectinesterase, triacylglycerol lipase, xylanase, beta-xylosidase or any combination thereof. Non-limiting examples of a flavoring include, for example, a floral, berry, nutty, caramel, chocolate, peppery, smoky, cheesy or meaty flavor, mints such as peppermint, citrus flavors such as orange and lemon, artificial vanilla, cinnamon and various fruit flavors. Non-limiting examples of an amino acid include, for example, alanine (Ala; A), arginine (Arg; R), asparagine (Asn; N), aspartic acid (aspartate, Asp; D), cysteine (Cys; C), glutamine (Gln; Q), glutamic acid (glutamate, Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V), and any combination thereof. Non-limiting examples of a preservative include, for example, sodium sorbate, potassium sorbate, sodium benzoate and potassium benzoate, and combinations thereof.

In some embodiments, the furoguaiacin or furoguaiacin-like compounds or guaiacin or guaiacin-like compounds are obtained from a Machilus edulis, Persea fructifera, Machilus thunberghii, Cinnamomum philippense, Saurus cernuus, Myristica fragrans, Guaiacum sanctum or Guaiacum officinale plant or plant part, or an extract thereof. In particular embodiments, the furoguaiacin or furoguaiacin-like compounds or guaiacin or guaiacin-like compounds are obtained from heartwood or branches of a Guaiacum sanctum or Guaiacum officinale plant, or an extract thereof. In certain embodiments, the furoguaiacin or furoguaiacin-like compounds or guaiacin or guaiacin-like compounds are obtained from an extract from a plant or part thereof. In further embodiments, the extract is produced from heartwood, whole trunk, bark, stems, leaf, flowers, branches, roots or fruits of a plant or part thereof, or any combination thereof. In yet further embodiments, the extract is an aqueous, ethanolic, methanolic, isopropanolic, ethylacetate, acetonic, or hexane extract, or mixtures thereof, or a supercritical CO₂ extract. In some embodiments, the furoguaiacin or furoguaiacin-like compounds or guaiacin or guaiacin-like compounds are synthetically produced.

In additional embodiments, the furoguaiacin-like or guaiacin-like compounds have the chemical structure:

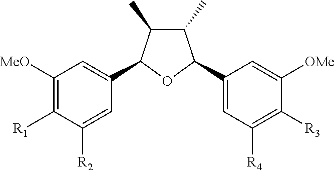

wherein R₁ is —OH, —OCH₃ or —OCH₂, R₂ is —OCH₃ or —O, R₃ is —OCH₂ or —O, and R₄ is —O or OCH₃, or the corresponding glycosidic forms

In particular embodiments, the furoguaiacin or furoguaiacin-like compounds or guaiacin or guaiacin-like compounds correspond to peaks with retention times of about 12.5 minutes, 16.5 minutes, 18.8 minutes, 20.5 minutes, 26.3 minutes, 26.8 minutes, 34.7 minutes, 35.6 minutes, 36.8 minutes, 39.1 minutes, 41.9 minutes, 43.9 minutes, 44.6 minutes, 45.0 minutes, 46.3 minutes, 48.2 minutes, 50.7 minutes, 53.5 minutes, 54.6 minutes, and 61.1 minutes on an HPLC chromatogram of an ethanol extract of heartwood or branches of a Guaiacum sanctum plant as detailed in Example 1 below. In other embodiments using different HPLC conditions (e.g., column material, solvents and/or flow rate) the major peaks might have different retention times, but these major peaks would still be present.

In other embodiments of a composition provided herein, lignans comprise between about 5% and about 30% of the composition, including, for example, at least about 7%, 9%, 11%, 13% 15% or 20% of the composition. In other embodiments, total lignans in said composition comprise at least 20% guaiacin or guaiacin-like compounds and furoguaiacin or furoguaiacin-like compounds, including at least about 30%, 40%, 50% or 60% of the total lignans. In particular embodiments, the guaiacin or guaiacin-like compounds make up from about 20% to about 60% of total lignans comprised in said composition by weight. In further embodiments, the furoguaiacin or furoguaiacin-like compounds make up from about 20% to about 60% of total lignans comprised in said composition by weight.

In still other embodiments, the present disclosure provides an animal feed comprising an animal feed ingredient composition described herein comprising an effective amount of furoguaiacin or furoguaiacin-like compounds or guaiacin or guaiacin-like compounds, or biologically acceptable salts thereof, in an amount effective to improve zootechnical performance in an animal fed the animal feed relative to a control feed lacking said animal feed ingredient composition. In certain embodiments, the animal feed is a chicken feed, pig feed, dairy cow feed, or beef cattle feed. In specific embodiments, the improved zootechnical performance is altered feed intake, increased average daily weight gain, increased feed efficiency, decreased feed conversion ratio or increased milk yield.

The term “feed conversion ratio” as used herein refers the amount of feed fed to an animal to increase the weight of the animal by a specified amount. An improved feed conversion ratio means a lower or decreased feed conversion ratio. By “lower or decreased feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise the feed ingredient or additive composition.

The term “feed efficiency” refers to the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without the feed ingredient or additive composition being present.

In various embodiments, the furoguaiacin or furoguaiacin-like compounds or guaiacin or guaiacin-like compounds, or biologically acceptable salts thereof, is present in an amount of from about 0.01 mg to about 2000 mg per kg of animal feed. In certain embodiments the furoguaiacin or furoguaiacin-like compounds or guaiacin or guaiacin-like compounds, or biologically acceptable salts thereof, is present in an amount of about 0.01, about 5, about 10, about 25, about 50, about 100, about 200, about 250, about 300, about 400, about 500, about 600, about 700, about 750, about 800, about 900, about 1000, about 1100, about 1200, about 1250, about 1300, about 1400, about 1500, about 1600, about 1700, about 1750, about 1800 or about 1900 or about 2000 mg per kg of animal feed. In other embodiments the furoguaiacin or furoguaiacin-like compounds or guaiacin or guaiacin-like compounds, or biologically acceptable salts thereof, is present in an amount of from about 0.01 mg to about 1500 mg, about 0.01 mg to about 1000 mg, about 0.01 mg to about 500 mg, about 0.01 mg to about 100 mg, about 0.01 mg to about 50 mg, about 0.01 mg to about 10 mg, about 0.01 mg to about 5 mg, about 0.01 mg to about 1 mg, about 0.01 mg to about 0.5 mg, about 0.01 mg to about 0.1 mg, or about 0.01 mg to about 0.05 mg per kg of animal feed. In still other embodiments the furoguaiacin or furoguaiacin-like compounds or guaiacin or guaiacin-like compounds, or biologically acceptable salts thereof, is present in an amount of from about 0.5 mg to about 2000 mg, about 1 mg to about 2000 mg, about 5 mg to about 2000 mg, about 10 mg to about 2000 mg, about 50 mg to about 2000 mg, about 100 mg to about 2000 mg, about 500 mg to about 2000 mg, about 1000 mg to about 2000 mg, or about 500 mg to about 2000 mg per kg of animal feed. In yet other embodiments the furoguaiacin or furoguaiacin-like compounds or guaiacin or guaiacin-like compounds, or biologically acceptable salts thereof, is present in an amount of from about 0.05 mg to about 1500 mg, about 1 mg to about 1000 mg, about 10 mg to about 500 mg, or about 50 mg to about 100 mg per kg of animal feed. In some embodiments, the furoguaiacin or furoguaiacin-like compounds or guaiacin or guaiacin-like compounds are obtained from a Machilus edulis, Persea fructifera, Machilus thunberghii, Cinnamomum philippense, Saurus cernuus, Myristica fragrans, Guaiacum sanctum or Guaiacum officinale plant or plant part, or an extract thereof. In other embodiments, the furoguaiacin or furoguaiacin-like compounds or guaiacin or guaiacin-like compounds are obtained from heartwood or branches of a Guaiacum sanctum or Guaiacum officinale plant, or an extract thereof.

In still another embodiment, the present disclosure provides a method of improving the performance of an animal feed comprising adding an effective amount of a composition comprising furoguaiacin or furoguaiacin-like compounds or guaiacin or guaiacin-like compounds as described herein, or biologically acceptable salts thereof, to the animal feed. In certain embodiments, the improved performance comprises altered feed intake, increased average daily weight gain, increased feed efficiency, decreased feed conversion ratio or increased milk yield in an animal fed said animal feed relative to a control animal feed to which the animal feed ingredient composition has not been added. In various embodiments, the animal feed is chicken feed, pig feed, dairy cow feed, or beef cattle feed.

In still yet another embodiment, the present disclosure also provides a method of improving the zootechnical performance of an animal comprising feeding to the animal an animal feed comprising an animal feed ingredient composition comprising an effective amount of furoguaiacin or furoguaiacin-like compounds or guaiacin or guaiacin-like compounds, or biologically acceptable salts thereof, in an amount effective to improve zootechnical performance in an animal fed the animal feed relative to a control feed lacking said animal feed ingredient composition. In some embodiments, the animal is a chicken, pig, dairy cow or beef cattle. In other embodiments, the improved zootechnical performance is altered feed intake, increased average daily weight gain, increased feed efficiency, decreased feed conversion ratio or increased milk yield.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 shows the HPLC chromatogram of GS heartwood. The numbers above the peaks are associated with the compounds as explained. Retention time for peaks in minutes shown in parentheses. Peaks 1 (5.519), 3 (15.576), 6 (19.474), and 8 (24.551): Vanillin-like; Peaks 2 (12.480) and 17 (43.972): Guaiaretic acid like; Peaks 4 (16.694), 9 (26.295), 10 (26.810), 11 (35.346), 12 (35.888), 13 (37.983), 14 (39.385), 16 (41.869), 19 (45.074), 21 (48.215) and 25 (54.615): Furoguaiacin-like; Peaks 5 (18.816), 7 (20.551), 18 (44.530), 20 (46.662), 23 (50.702), 24 (53.528) and 29 (67.062): Guaiacin-like; Peaks 15 (41.336), 22 (49.639), 26 (57.693), 27 (61.201), 28 (64.556) and 30 (68.034): Unknown compounds.

FIG. 2 shows the HPLC chromatogram of a branch of a mature tree (Tree stem diameter: 40 cm, branch diameter: 2.54 cm). The numbers above the peaks are associated with the compounds as explained. Retention time for peaks in minutes shown in parentheses. Peaks 1 (5.513), 3 (15.597), 6 (19.493) and 8 25.332): Vanillin-like; Peaks 2 (12.476) and 17 (43.981): Guaiaretic acid-like; Peaks 4 (16.112), 9 (26.340), 10 (26.872), 11 (33.806), 12 (35.386), 13 (36.421), 14 (37.887), 16 (41.887), 19 (43.861), 21 (48.223) and 25 (54.511): Furoguaiacin-like; Peaks 5 (18.840), 7 (20.581), 18 (44.586), 20 (45.090), 23 (50.674), 24 (53.558) and 29 (67.079): Guaiacin-like; Peaks 15 (41.372), 22 (49.445), 26 (57.715), 27 (61.268), 28 (64.576) and 30 (68.015): unknown compounds.

FIG. 3 shows the total lignans content, expressed in %, of different botanical parts of Guaiacum sanctum.

FIG. 4 shows the content in Furoguaiacin-like, Guaiacin-like and Guaiaretic acid-like in heartwood, branches, sapwood and bark of Guaiacum sanctum. The content is expressed in % of total lignans.

FIG. 5 illustrates that the dietary inclusion of 8 ppm of Total Lignans standardized in Furoguaiacin-like compounds and Guaiacin-like compounds (Diet 3-50% Furoguaiacin-like compounds and 40% Guaiacin-like compounds) improve feed conversation in chickens compared to a negative control and a positive control. The negative control is the diet with no feed additive (Diet 1). The positive control is the same diet with inclusion of a technology considered as a standard for alternative to antibiotic growth promoters (Diet 2-100 ppm of XTRACT® Evolution-B, Code X60-6930). Values shown in the figure are averages. In the figure, different letters on the bars indicate significant differences.

FIG. 6 shows the effect on standard doses of Total Lignans standardized in Furoguaiacin-like compounds and Guaiacin-like compounds on the production of an inflammatory cytokine, TNFα. The negative control is shown by the culture medium. The positive control is the inclusion of 1 μM of dexamethasone. Values shown in the figure are averages. The bars also show the standard deviation.

FIG. 7 shows the effect on standard doses of Total Lignans standardized in Furoguaiacin-like compounds and Guaiacin-like compounds on the production of an inflammatory cytokine, IL6. The negative control is shown by the culture medium. The positive control is the inclusion of 1 μM of dexamethasone. Values shown in the figure are averages. The bars also show the standard deviation.

FIG. 8 shows the effect on standard doses of Total Lignans standardized in Furoguaiacin-like compounds and Guaiacin-like compounds on the production of an inflammatory cytokine, IL1β. The negative control is showed by the culture medium. The positive control is the inclusion of 1 uM of dexamethasone. Values shown in the figure are averages. The bars also show the standard deviation.

FIG. 9 illustrates that standard doses of Total Lignans standardized in Furoguaiacin-like compounds and Guaiacin-like compounds on PGE2 production in chondrocytes in a cell-based bioassay in vitro. The black bar and hatched bars are the control, and the experimental treatments, respectively. Standard deviations are shown in the bars.

FIG. 10 illustrates that standard doses of Total Lignans standardized in Furoguaiacin-like compounds and Guaiacin-like compounds on matrix metalloproteinase-3 in chondrocytes in a cell-based bioassay in vitro. The black bar and hatched bars are the control, and the experimental treatments, respectively. Standard deviations are shown in the bars.

### DETAILED DESCRIPTION

The present disclosure provides a feed additive for domesticated animals that is capable of increasing feed performance, as well as associated methods for promoting growth and zootechnical performance in domesticated animals. The animal feed additive provided herein contains Total Lignans standardized in the contents of Furoguaiacin-like compounds and Guaiacin-like compounds. The present disclosure also describes methods for improving growth via the dietary inclusion of Total Lignans standardized in Furoguaiacin-like compounds and Guaiacin-like compounds.

Phenolic compounds are a class of plant secondary metabolites with important roles in plant physiology. The structure of phenolic compounds varies extensively and according to their structures, they are classified in 4 categories: phenolic acids, flavonoids, stilbenoids, and lignans. Lignans are widely distributed throughout plants such as in cereals, grains, berries and garlic. Total Lignans refers to the sum of the lignans in a plant composition.

The instant disclosure provides a novel approach using total lignans, standardized using two specific lignan constituents, Furoguaiacin-like compounds and Guaiacin-like compounds, for improving performance (increased intake, daily gains and/or improved efficiency) in farm or other domesticated animals. These compounds may be used during normal rearing conditions.

The word lignan encompasses a variety of chemical structures. Pilkington classified lignans in two main categories: classical lignans and neolignans, and two smaller categories: flavonolignans and coumarolignans (Lignans: A Chemometric Analysis. Molecules 23:1666, 2018). Each of these categories are organized in several subcategiories. There are 6 main subcategories of classical lignans: CL1: dibenzylbutanes, CL2: dibenzylbutyrolactones, CL3: arylnaphthalenes/aryltetralins, CL4: dibenzocyclooctadienes, CL5: substituted tetrahydrofurans, and CL6: 2,6-diarylfurofurans. There are 15 subcategories of neolignans, with the most common subcategories being: NL1: benzofurans, NL2: 1,4-benzodioxanes, NL3: alkyl aryl ethers, NL4: biphenyls, NL5: cyclobutanes, NL6: 8-10-bicyclo[3.2.1]octanes, NL7: 8-30-bicyclo[3.2.1]octanes and NL8: biphenyl ethers.

The chemical structure of a natural molecule correlates with its function. Molecules with different chemical structures show different biological or physiological functions between categories and even subcategories. Lignans from different lignan categories and subcategories will show different physiological functions and impact, and hence, different productive performance on the target animal. In other words, the physiological function, and hence, productive performance response of an animal, of a lignan correlates with its chemical structure.

Furoguaiacin (aka alpha-Guaiaconic acid, structure 1) is a classical lignan belonging to the substituted tetrahydrofuran subcategory (see CL5 subcategory above) with the molecular formula C₂₀H₂₀O₅.

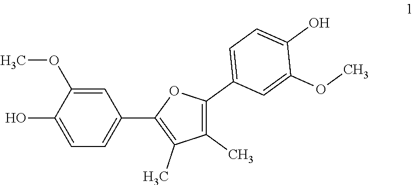 [SMILES: COc1cc(-c2oc(-c3ccc(O)c(OC)c3)c(C)c2C)ccc1O]

Guaiacin (structure 2) is a classical lignan belonging to the arylnaphthalene/aryltetraline subcategory of lignan (see CL3 subcategory above) with the molecular formula C₂₀H₂₄O₄.

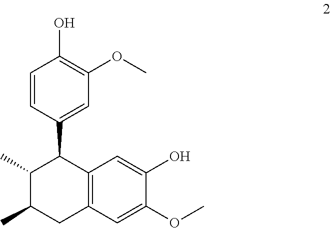

Guaiacum sanctum or Guaiacum officinale are one source for Guaiacin or guaiacin-like compounds. Guaiacum gum or guaiac resin can be extracted from the heartwood of Guaiacum officinale and Guaiacum sanctum. Furoguaiacin or furoguaiacin-like compounds and Guaiacin or guaiacin-like compounds are mainly found in Guaiacum trees or their extracts, although other sources of furoguaiacin or furoguaiacin-like compounds and guaiacin or guaiacin-like compounds include, but are not limited to, Machilus edulis, Persea fructifera, Machilus thunberghii, Cinnamomum philippense, Saurus cernuus, and Myristica fragrans.

Guaiacin-Like and Furoguaiacin-Like Compounds

Furoguaiacin-like and guaiacin-like compounds in certain embodiments can be defined herein by the core structure shown below (structure 3):

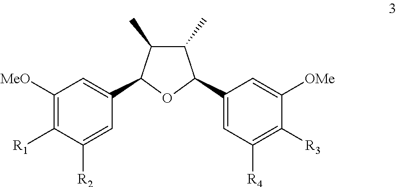

In certain embodiments, the R₁, R₂, R₃ and R₄ groups are as shown in Table 1, below.

TABLE 1

| R₁ | R₂ | R₃ | R₄ |
|---|---|---|---|
| OH | OCH₃ | OCH₂ | O |
| OCH₃ | OCH₃ | OCH₂ | O |
| OCH₃ | OCH₃ | OH | OCH₃ |
| OCH₂ | O | OCH₂ | O |

In additional embodiments the furoguaiacin-like compounds and guaiacin-like compounds include the corresponding glycosidic forms.

Animal Feed Components

Provided herein are novel animal feed compositions with improved performance as a result of comprising the animal feed ingredient composition described herein. Different domesticated animals have different feed requirements. For example, the main ingredients in chicken feed are generally cereals, including, but not limited to, wheat, corn, sorghum, oats, barley or rye, protein, which can come from oilseed meals, and fat or oil. However, other ingredients can be added to chicken feed, including, but not limited to, a source of calcium, salts, minerals, probiotics, vitamins, amino acids, flavorings and preservatives. An exemplary chicken feed for chicks and pullets includes protein, lysine, methionine, fat, fiber, calcium, phosphorous, NaCl, manganese, vitamin A and vitamin E.

The main ingredients in pig feed are generally cereals, including, but not limited to, rice bran, broken rice, and corn, protein, which can come from oilseed meals such as alfalfa meal, or soybean meal, minerals and vitamins. However, other ingredients can be added to pig feed, including, but not limited to, a source of calcium, salts, minerals, probiotics, vitamins, amino acids, flavorings and preservatives. An exemplary pig feed includes an energy source (cereals), protein, vitamins, minerals, fiber, prebiotics and botanicals.

The main ingredients in dairy cattle feed are generally cereals, protein, which can come from oilseed meal such as cottonseed meal or soybean meal, sugar and fat. However, other ingredients can be added to dairy cattle feed, including, but not limited to, fiber, a source of calcium, salts, minerals, probiotics, vitamins, amino acids, flavorings and preservatives.

The main ingredients in beef cattle feed are generally cereals, including, but not limited to, wheat, corn, sorghum, oats, barley or rice, and protein, which can come from de-oiled rice bran, rice polish, wheat bran or corn bran. However, other ingredients can be added to beef cattle feed, including, but not limited to, fiber, fat, salts, minerals, probiotics, vitamins, amino acids, flavorings and preservatives.

The main ingredients in sheep feed are generally cereals, such as alfalfa and corn, vitamins, such as vitamin A, vitamin D and vitamin E, selenium, mineral salts and phosphorous. However, other ingredients can be added to sheep feed, including, but not limited to, fat, probiotics, amino acids, flavorings and preservatives. An exemplary sheep feed for mature ewes and rams includes cereals, protein, fat, fiber, calcium, ammonium chloride, phosphorous, NaCl, selenium and vitamin A.

The main ingredients in goat feed are generally cereals, such as hay, alfalfa, barley corn and oats, protein, which can come from distilled grains and meals, fat, fiber and minerals such as calcium, phosphorous, NaCl, copper, selenium, and vitamins such as vitamin A, vitamin D and vitamin E. However, other ingredients can be added to goat feed, including, but not limited to, probiotics, amino acids, flavorings and preservatives. An exemplary goat feed includes grain products, protein, fat, fiber, acid detergent fiber and calcium, phosphorous, NaCl, as well as copper, selenium, and vitamins such as vitamin A, vitamin D and vitamin E.

As described herein, the performance of any such animal feeds may be improved by the addition of the animal feed ingredient provided by the present disclosure. The amount added may be optimized depending upon the type of feed, animal physiology, conditions under which the animal being fed is raised, and other conditions as will be understood to those of skill in the art according to the teachings of the present disclosure.

### EXAMPLES

### Example 1

In this example, different botanical parts (heartwood, bark, sapwood, branches of 2.5 cm of diameter and leaves) of the same plant (Guaiacum sanctum) were evaluated for their Total Lignans, Furoguaiacin and Guaiacin contents.

Plant samples were ground to a fine powder and extracted with 80% v/v ethanol for 2×15 min in an ultrasonic bath using a drug:solvent ratio of 1:50. The crude extract was centrifuged for 5 min at 14,500 rpm and the supernatant was used directly for High Performance Liquid Chromatography (HPLC) analysis.

The HPLC system was equipped with an autosampler and a diode array detector. As stationary phase a column packed with octadecylsilyl silica gel for chromatography (e.g., Zorbax SB—C18 4.6*250 mm, 5 μm) was used. The mobile phases consisted in Solvent A (5% acetic acid) and solvent B (acetonitrile/methanol 50/50 (v/v)). A linear gradient was applied with a flow rate of 1.5 ml/min. First, detection of the lignan peaks was carried out at 220, 280 and 320 nm. For the determination of the Total Lignans content, all lignan peaks in the chromatograms were identified by their UV-spectra which consist of either two or three maxima values at 238, 280 and at 230, 280 and 320 nm. For the quantification, the sum of the lignan peak areas was calculated by using nordihydroguaiaretic acid (NDGA) as external standard, since the UV spectrum of NDGA is like the spectra of Guaiacum lignans. As reference compounds, NDGA (Sigma Aldrich, art. No. 74540), guaiacin (ambinter, c/o Greenpharma; No. amb35820270) and vanillin (Sigma Aldrich, No. V1104) were used. Of all relevant peaks in the HPLC chromatograms, the UV spectrum was determined. Obtained UV spectra were then compared to known UV spectra, i.e., spectra published in the literature or spectra obtained with the reference compounds. Based on similarities of the spectra, peak identification or at least the allocation to specific substance classes was carried out. To verify UV spectrum similarity, the spectra of the peaks were overlaid with the spectrum of the reference compounds (vanillin, guaiacin). The degree of similarity was then calculated by the HPLC software. Quantification of the lignans was carried out using NDGA as reference compound.

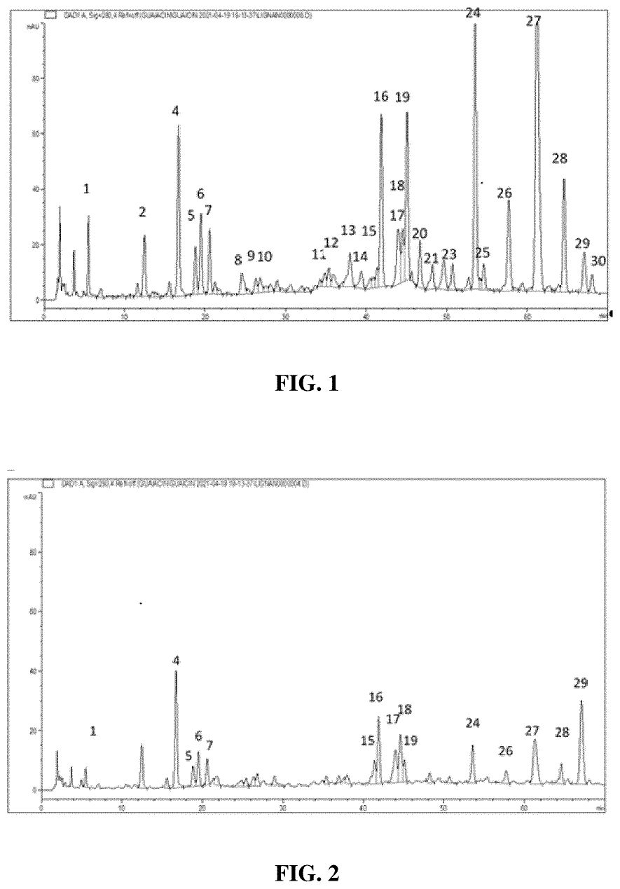

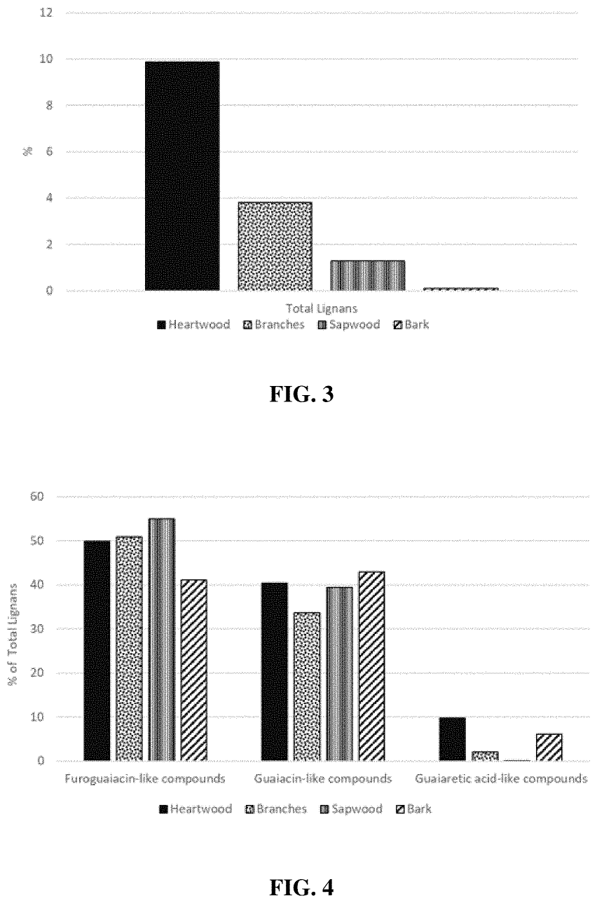

The results showed the composition of the lignan fraction of different botanical parts of Guaiacum sanctum. For example, the HPLC chromatograms of FIG. 1 and FIG. 2 show the lignan composition in the heartwood and branches, respectively. FIG. 3 shows the total lignans concentrations of the botanical parts of Guaiacum sanctum. The concentration in the branches is lower by a factor of 2-3 compared to the heartwood, but still higher than in the other Guaiacum sanctum parts (sapwood, bark); no relevant lignan content was observed in the leaves. Finally, FIG. 4 presents the composition of the total lignans fraction in terms of Furoguaiacin-like compounds, Guaiacin-like compounds and Guaiaretic acid-like compounds. Surprisingly, the lignan profile in the branches is quite similar to the one found in the heartwood.

### Example 2

This example is used to illustrate the total Lignans standardized with Furoguaiacin-like compounds and Guaiacin-like compounds on performance of farm animals.

The study evaluated the effect of increasing dietary inclusion of total lignans standardized in Furoguaiacin-like compounds and Guaiacin-like compounds when fed to broiler chickens from 1 to 28 days of age on growth performance, feed intake and feed efficiency (measured by feed conversion ratio). Treatments included two control diets and one experimental diet.

The basal diet composition was the following (expressed in kg/100 kg): corn 35.00, Oats 14.00, Barley 7.00, vegetable oil 6.00, corn gluten meal 1.00, Soybean meal 29.00, rapeseed meal 3.00, monocalcium phosphate 1.80, limestone 0.40, NaCl 0.40, Lysine 0.50, Methionine 0.60 and a premixture containing other minerals and vitamins 0.500. The nutritional composition of Diet 1 was 12.878 MJ/kg, crude protein 22.094%, crude fat 8.681%, fiber 4.203%, Lysine 1.559%, Methionine 0.7907%.

The treatments were obtained by supplementing the basal diet with a compound. Diet 1 acted as a negative control and did not include any compound. Diet 2 acted as a positive control diet; it included 100 g per metric ton of XTRACT® Evolution-B, Code X60-6930, a product accepted as an alternative to antibiotic growth promoter (EFSA Journal, 2015). Diet 3 was Diet 1 supplemented with 8 g per metric ton of Total Lignans composed of 50% Furoguaiacin-like compounds and 40% Guaiacin-like compounds.

Approximately 400 1-day-old male Ross 308 chicks were obtained from a commercial hatchery. On arrival, 400 chicks were allocated to 20 floor pens, twenty birds in each pen. Each of the pens has a solid floor with an area of 150×140 cm that was covered with a bedding material (wood shavings). Birds received one of the 3 experimental diets. Feed and water were offered ad libitum to birds throughout the experiment.

Each of the 3 treatments (diets 1 to 3 described above) was offered to birds in 8 pens in a randomized block design. Information on growth and feed intake were obtained from day old to 28 days of age. The room temperature was approximately 32° C., at day old, and was gradually reduced to 20° C. at the end of the 21-day feeding period. A standard lighting program for broilers was used. The birds were weighed at the beginning (at day old), at 14-day age and at the end (28 day old) of the study, and the weight gain and feed efficiency were determined.

The results are summarized in FIG. 5. Feed efficiency was estimated using the feed conversion ratio. The main results are that: 1) The inclusion of 100 g per metric ton of XTRACT® Evolution-B, Code X60-6930 improved feed efficiency in a magnitude similar to published information on this product. For example, Bravo et al. (J. Appl. Poul. Res. 20:115-120, 2009) showed that the addition of the same dose of the same product on chickens improved feed efficiency by 2.28% (feed conversion: 1.75 vs. 1.79 at 34 days of age). In the present study, the same product improved feed efficiency by 2.2% (feed conversion: 1.60 vs. 1.57 at 28 days of age); and 2) The inclusion of 8 ppm of Total Lignans composed of 50% Furoguaiacin-like compounds and 40% Guaiacin-like compounds significantly improved the performance of chickens. Feed efficiency was improved by 4.7% (feed conversion: 1.52 vs. 1.60).

### Example 3

In this example, the effect of increasing dose of Total Lignans standardized on Furoguaiacin-like compounds and Guaiacin-like compounds on anti-inflammatory cytokines in vitro is described. The anti-inflammatory effect, i.e., the inhibitory effect on cytokine release of a 70% v/v ethanolic Guaiacum sanctum extract, standardized in Total Lignans, Furoguaiacin and Guaiacin was determined using the THP-1 human monocytic cell line which were differentiated into macrophage-like cells by phorbol myristate acetate (PMA). Prior to the assay the potential cytotoxic effect of the test compounds was determined using the MTT (3-(4,5-dimethylthiazol-2-yl)-2,5-diphenyltetrazolium bromide) assay for determining mitochondrial dehydrogenase activities in the living cells. The anti-inflammatory effect on cytokine release in the THP-1 macrophage cells was measured by incubating the cells with three concentrations of the guaiac extract followed by the addition of LPS to stimulate the cytokine production. After 24 h the amount of TNF-alpha, IL-1 beta and IL-6 was determined in the supernatant of the cell cultures by capture ELISA using commercial kits.

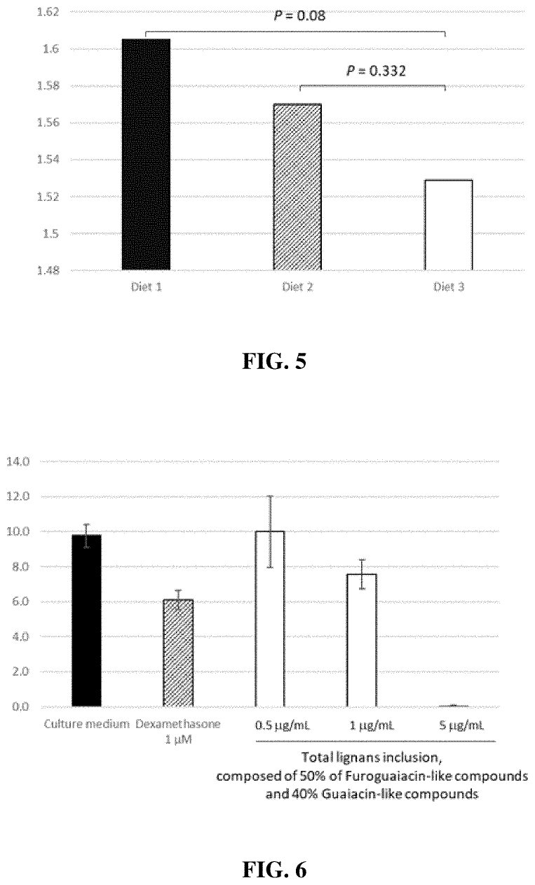

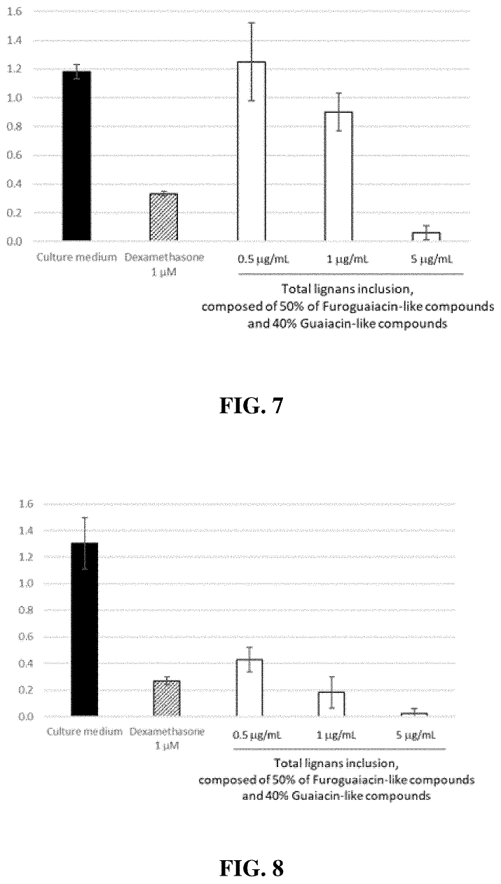

The main results are: 1) Increasing doses of Total Lignans standardized on Furoguaiacin-like compounds and Guaiacin-like compounds reduced the production of the inflammatory cytokine TNFα (see FIG. 6); 2) Increasing doses of Total Lignans standardized on Furoguaiacin-like compounds and Guaiacin-like compounds reduced the production of the inflammatory cytokine IL6 (see FIG. 7); and 3) Increasing doses of Total Lignans standardized on Furoguaiacin-like compounds and Guaiacin-like compounds reduced the production of the inflammatory cytokine IL1β (see FIG. 8).

### Example 4

In this example, the effect of increasing dose of Total Lignans standardized on Furoguaiacin-like compounds and Guaiacin-like compounds on markers of osteoarthritis was evaluated in an in vitro cell based assay.

Chondrocytes were seeded in 96-well plates and cultured for 24 hours in culture medium. The culture medium was composed of Dulbecco's modified Eagle's medium supplemented with 4 mM L-glutamine, 50 U/mL Penicillin and 50 μg/mL Streptomycin, 10% fetal calf serum. The medium was then removed and replaced by assay medium containing or not (control) the test compounds or the references (dexamethasone at 0.1 μM for MMP-3 and indomethacin at 1 μM for PGE2) and the cells were pre-incubated for 24 hours. The assay medium was composed of Dulbecco's modified Eagle's medium supplemented with 4 mM L-glutamine, 50 U/mL Penicillin and 50 μg/mL Streptomycin, 2% fetal calf serum. After the pre-incubation, the medium was removed and replaced by assay medium containing or not (control) the test compounds or the references in presence of the mix of cytokines (IL-1β+TNF-α+IFN-γ+IL-6 at 3 ng/ml each) and the cells were incubated for 48 hours. A non-stimulated control was performed in parallel. All experimental conditions were performed in n=3.

At the end of incubation, the supernatants were collected for the quantification of the release of an inflammatory marker Prostaglandin E2 (PGE2) as well as the level of a matrix-degrading enzyme (MMP-3, a metalloprotease involved in cartilage degradation) were determined using specific ELISA kits according to the supplier's instructions. The assay kits were Enzo Life Sciences, Ref. ADI-901-001 (for PGE2) and R&D Systems, Ref. DY513 (for MMP-3).

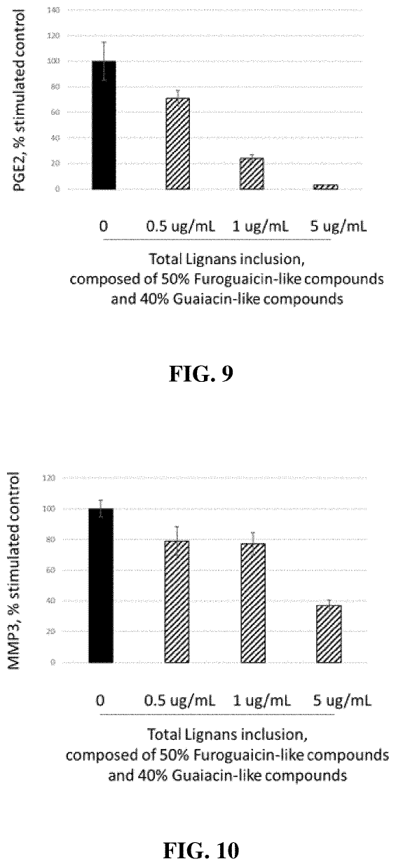

The results were the following. Increasing doses of Total Lignans standardized on Furoguaiacin-like compounds and Guaiacin-like compounds (0.5, 1 and 5 μg/mL) decreased PGE2 production (FIG. 9) and reduced matrix metalloproteinase-3 (FIG. 10).

To facilitate the understanding of this disclosure, a number of terms are defined below. Terms defined herein have meanings as commonly understood by a person of ordinary skill in the areas relevant to the present disclosure. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. For example, all of the disclosed components of the preferred and alternative embodiments are interchangeable providing disclosure herein of many systems having combinations of all the preferred and alternative embodiment components. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. All such similar substitutes and modifications apparent to those skilled in the art are deemed to be within the spirit, scope and concept of the disclosure as defined by the appended claims.

1. An animal feed ingredient composition comprising an effective amount of furoguaiacin or a furoguaiacin-like compound or guaiacin or a guaiacin-like compound, or a biologically acceptable salt thereof, wherein the effective amount improves the performance of an animal feed to which the ingredient is added.

2. The composition of claim 1 further defined as comprising at least one animal feed component selected from the group consisting of a vitamin, mineral, probiotic, enzyme, flavoring, amino acid, and preservative.

3. The composition of claim 1, wherein the furoguaiacin or furoguaiacin-like compound or guaiacin or guaiacin-like compound;
a) is obtained from a Machilus edulis, Persea fructifera, Machilus thunberghii, Cinnamomum philippense, Saurus cernuus, Myristica fragrans, Guaiacum sanctum or Guaiacum officinale plant or plant part, or an extract thereof;
b) is obtained from an extract from a plant or part thereof;
c) is synthetically produced; or
d) corresponds to peaks with retention times of about 12.5 minutes, 16.5 minutes, 18.8 minutes, 20.5 minutes, 26.3 minutes, 26.8 minutes, 34.7 minutes, 35.6 minutes, 36.8 minutes, 39.1 minutes, 41.9 minutes, 43.9 minutes, 44.6 minutes, 45.0 minutes, 46.3 minutes, 48.2 minutes, 50.7 minutes, 53.5 minutes, 54.6 minutes, and 61.1 minutes on an HPLC chromatogram of an ethanol extract of heartwood or branches of a Guaiacum sanctum plant.

4. The composition of claim 3, wherein the furoguaiacin or furoguaiacin-like compound or guaiacin or guaiacin-like compound is obtained from heartwood or branches of a Guaiacum sanctum or Guaiacum officinale plant, or an extract thereof.

5. (canceled)

6. The composition of claim 3, wherein the extract;
a) is produced from heartwood, whole trunk, bark, stems, leaf, flowers, branches, roots or fruits of a plant or part thereof, or any combination thereof; or
b) is an aqueous, ethanolic, methanolic, isopropanolic, ethylacetate, acetonic, hexane, or a mixture thereof, or a supercritical CO₂ extract.

7.-8. (canceled)

9. The composition of claim 1, wherein the furoguaiacin-like compound or guaiacin-like has the chemical structure: 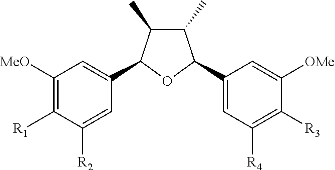
wherein R₁ is —OH, —OCH₃ or —OCH₂, R₂ is —OCH₃ or —O, R₃ is —OCH₂ or —O, and R₄ is —O or —OCH₃, and the corresponding glycosidic forms

10. (canceled)

11. The composition of claim 1, wherein:
a) lignans comprise between about 5% and about 30% of the composition; or
b) total lignans in the composition comprise at least 20% furoguaiacin or furoguaiacin-like compounds and guaiacin or guaiacin-like compounds.

12. The composition of claim 11, wherein lignans comprise at least 9% of the composition.

13. (canceled)

14. The composition of claim 1, further wherein:
a) the guaiacin or guaiacin-like compound makes up from about 20% to about 60% of total lignans comprised in said composition by weight; or
b) the furoguaiacin or furoguaiacin-like compound makes up from about 20% to about 60% of total lignans comprised in the composition by weight.

15. (canceled)

16. An animal feed comprising the animal feed ingredient composition of claim 1 in an amount effective to improve zootechnical performance in an animal fed the animal feed relative to a control feed lacking the animal feed ingredient composition.

17. The animal feed of claim 16, wherein:
a) the animal feed is chicken feed, pig feed, dairy cow feed, or beef cattle feed; or
b) the improved zootechnical performance is altered feed intake, increased average daily weight gain, increased feed efficiency, decreased feed conversion ratio or increased milk yield.

18. (canceled)

19. The animal feed of claim 16, wherein the furoguaiacin or furoguaiacin-like compound or guaiacin or guaiacin-like compound, or biologically acceptable salt thereof, is present in an amount of from about 0.01 mg to about 2000 mg per kg of animal feed.

20. The animal feed of claim 16, wherein the furoguaiacin or furoguaiacin-like compound or guaiacin or guaiacin-like compound is obtained from a Machilus edulis, Persea fructifera, Machilus thunberghii, Cinnamomum philippense, Saurus cernuus, Myristica fragrans, Guaiacum sanctum or Guaiacum officinale plant or plant part, or an extract thereof.

21. The animal feed of claim 16, wherein the furoguaiacin or furoguaiacin-like compound or guaiacin or guaiacin-like compound is obtained from heartwood or branches of a Guaiacum sanctum or Guaiacum officinale plant, or an extract thereof.

22. A method of improving the performance of an animal feed, comprising adding an effective amount of the composition of claim 1 to the animal feed.

23. The method of claim 22, wherein:
a) the improved performance comprises altered feed intake, increased average daily weight gain, increased feed efficiency, decreased feed conversion ratio or increased milk yield in an animal fed the animal feed relative to a control animal feed to which the animal feed ingredient composition has not been added; or
b) the animal feed is chicken feed, pig feed, dairy cow feed, or beef cattle feed.

24. (canceled)

25. The method of claim 22, wherein the furoguaiacin or furoguaiacin-like compound or guaiacin or guaiacin-like compound, or biologically acceptable salt thereof, is present in an amount of from about 0.01 mg to about 2000 mg per kg of animal feed.

26. A method of improving the zootechnical performance of an animal comprising feeding the animal feed of claim 16 to the animal.

27. The method of claim 26, wherein:
a) the animal is a chicken, pig, dairy cow, or beef cattle; or
b) the improved zootechnical performance is altered feed intake, increased average daily weight gain, increased feed efficiency, decreased feed conversion ratio or increased milk yield.

28. (canceled)

29. The method of claim 26, wherein the animal feed comprises the furoguaiacin or furoguaiacin-like compound or guaiacin or guaiacin-like compound, or biologically acceptable salt thereof, in an amount of from about 0.01 mg to about 2000 mg per kg of animal feed.
